(19) United States
(12) Patent Application Publication
(10) Pub. No.: US 2021/0317424 A1
(43) Pub. Date: Oct. 14, 2021

(54) METHOD AND KIT FOR TEMPLATE-INDEPENDENT NUCLEIC ACID SYNTHESIS

(71) Applicant: Cheng-Yao CHEN, Hsinchu (TW)

(72) Inventor: Cheng-Yao CHEN, Hsinchu (TW)

(21) Appl. No.: 17/354,543

(22) Filed: Jun. 22, 2021

Related U.S. Application Data
(63) Continuation-in-part of application No. 16/725,420, filed on Dec. 23, 2019.

(51) Int. Cl. C12N 9/12; C07H 21/04

(52) U.S. Cl. CPC C12N 9/1252; C07H 21/04

(57) ABSTRACT

A method for synthesizing a nucleic acid includes providing an initiator having an unprotected nucleoside base and a 3′ hydroxyl group at a 3′ terminus, providing a nucleic acid polymerase having at least one conservative catalytic polymerase domain of a family-B DNA polymerase, providing at least one nucleotide monomer, and exposing the initiator to the nucleotide monomer in the presence of the nucleic acid polymerase, at a temperature ranging from 20° C. to 90°, and in the absence of a template, such that the nucleotide monomer is incorporated to the initiator to form the nucleic acid. Also disclosed is a kit includes the initiator, the nucleic acid polymerase, and the nucleotide monomer, and is used according to the method.

### CROSS-REFERENCE TO RELATED APPLICATION

This application is a continuation-in-part of U.S. patent application Ser. No. 16/725,420, filed on Dec. 23, 2019, the entire content of which is incorporated by reference.

### FIELD

The disclosure relates to a method and a kit for nucleic acid synthesis, more particularly to a method and a kit for template-independent nucleic acid synthesis.

### BACKGROUND

De novo DNA synthesis dispensing with a DNA template has been developed during past decades. Among the currently available template-independent DNA synthesis methods, the phosphoramidite-based chemical DNA synthesis has been well-known since early 1980's, but basically has remained unchanged since then. The phosphoramidite-based chemical DNA synthesis requires four consecutive reaction steps, including de-blocking, coupling, capping, and oxidation steps, to add one nucleoside to another nucleoside tethered to a solid support. However, one of the major drawbacks of the phosphoramidite-based chemical DNA synthesis is inevitable use of hazardous chemicals in the aforesaid reaction steps.

Due to growing demand for environmental protection, green technology applicable to DNA synthesis has drawn attention of researchers. Therefore, enzymatic DNA synthesis, which can greatly reduce use of hazardous chemicals, seems promising since such synthesis has merits such as longer strand generation, a lower error rate, a faster cycle time, a lower production cost, etc.

Speaking of template-independent enzymatic DNA synthesis, terminal deoxynucleotidyl transferase (TdT) has been found to be a template-independent DNA polymerase that adds all four deoxynucleoside triphosphates (dNTPs) to the 3′ termini of normal DNA strands. TdT is currently the preferred enzyme for template-independent DNA synthesis, whereas its intrinsic properties present a barrier in application such as thermal instability and random nucleotide incorporation.

Specifically, TdT belongs to the X Family of low-fidelity DNA polymerases and is only present in mesophilic organisms with minimal thermostability. The TdT-based DNA synthesis requires only two reaction steps, namely, a single-nucleotide addition by TdT and subsequent removal of the 3′-protective group from the extended 3′-end of the single-stranded DNA strand being synthesized. Even though TdT and its homologs have been widely applied to numerous, enzymatic DNA synthesis platforms, the Tdt-based template-independent DNA synthesis can be hardly commercialized due to unsatisfactory synthesis efficiency, short product length, long synthesis cycle time, and so forth. Accordingly, lots of efforts are made on engineering TdT to optimize the DNA synthesis functionality. However, the progress seems to be sluggish. Thus, there is a need for more efficient, stable, and reliable methods of enzymatic nucleic acid synthesis.

### SUMMARY

Therefore, the first objective of the disclosure is to provide a method and a kit for synthesizing a nucleic acid, which can alleviate at least one of the drawbacks of the prior art.

The method includes providing an initiator having a 3′ end having an unprotected hydroxyl group, providing a nucleic acid polymerase having at least a conservative catalytic polymerase domain of a family-B DNA polymerase, providing a nucleotide monomer, and exposing the initiator to the nucleotide monomer in the presence of the nucleic acid polymerase and a metal cofactor which is a bivalent cation, and in the absence of a template, such that the nucleotide monomer is incorporated to the initiator.

The kit includes an initiator as described above, a nucleic acid polymerase as described above, and a nucleotide monomer as described above. The kit is used according to a method as described above.

The second objective of the disclosure is to provide another method and another kit for synthesizing a nucleic acid, which can alleviate at least one of the drawbacks of the prior art.

The another method for synthesizing a nucleic acid includes providing an initiator having an unprotected nucleoside base and a 3′ hydroxyl group at a 3′ terminus, providing a nucleic acid polymerase having at least one conservative catalytic polymerase domain of a family-B DNA polymerase, providing at least one nucleotide monomer, and exposing the initiator to the at least one nucleotide monomer in the presence of the nucleic acid polymerase, at a temperature ranging from 20° C. to 90°, and in the absence of a template, such that the at least one nucleotide monomer is incorporated to the initiator to form the nucleic acid.

The another kit includes an initiator, a nucleic acid polymerase, and a nucleotide monomer as described above in the another method. The kit is used according to the another method.

### BRIEF DESCRIPTION OF THE DRAWINGS

Other features and advantages of the disclosure will become apparent in the following detailed description of the embodiments with reference to the accompanying drawings, of which:

FIG. 1 represents the de novo nucleic acid synthesis scheme using family-B DNA polymerase;

FIG. 2 is an image of denaturing urea-polyacrylamide gel showing products of template-independent nucleic acid synthesis obtained at different temperatures using KOD1^{exo-} DNA polymerase, in which the symbol “S” indicates the size and position of initiator DNA;

FIG. 3 is an image of denaturing urea-polyacrylamide gel showing products of template-independent nucleic acid synthesis obtained at different temperatures using Vent^{exo-} DNA polymerase, in which the symbol “S” indicates the size and position of initiator DNA;

FIG. 4 is an image of denaturing urea-polyacrylamide gel showing products of template-independent nucleic acid synthesis obtained at different temperatures using Pfu^{exo-} DNA polymerase, in which the symbol “S” indicates the size and position of initiator DNA;

FIG. 5 is an image of denaturing urea-polyacrylamide gel showing products of template-independent nucleic acid synthesis obtained at temperature increment ranging from 30° C. to 75° C. using KOD1^{exo-} DNA polymerase, in which the symbol “S” indicates the size and position of initiator DNA;

FIG. 6 is an image of denaturing urea-polyacrylamide gel showing results of template-independent nucleic acid synthesis obtained at temperature increment ranging from 45° C. to 90° C. using the conventional, template-independent terminal deoxynucleotidyl transferase (TdT), in which the symbol “S” indicates the size and position of initiator DNA;

FIG. 7 is an image of denaturing urea-polyacrylamide gel showing products of template-independent nucleic acid synthesis obtained at temperature increment ranging from 30° C. to 75° C. using Vent^{exo-} DNA polymerase, in which the symbol “S” indicates the size and position of initiator DNA; and

FIG. 8 is an image of denaturing urea-polyacrylamide gel showing products of template-independent nucleic acid synthesis obtained at temperature increment ranging from 30° C. to 75° C. using Pfu^{exo-} DNA polymerase, in which the symbol “S” indicates the size and position of initiator DNA.

### DETAILED DESCRIPTION

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The practice of the disclosure will employ, unless otherwise specified, conventional techniques of molecular biology, microbiology and recombinant DNA techniques, which are within the skill of the art. Such techniques are described fully in the literature and are not detailed herein.

References and citations to other documents, such as patents, patent applications, patent publications, journals, papers, and articles in this disclosure are hereby incorporated herein by reference in their entirety for all purposes. The terminology of several essential terms is provided herein to explain the present disclosure more clearly without any intention to limit the scope of the claimed subject matter, and the definition and/or description set forth herein prevails over the definition that is incorporated by reference.

The terms “nucleic acid”, “nucleic acid sequence”, and “nucleic acid fragment” as used herein refer to a deoxyribonucleotide or ribonucleotide sequence in single-stranded or double-stranded form, of which the sources are not limited herein; and generally, includes naturally occurring nucleotides or artificial chemical mimics. The term “nucleic acid” as used herein is interchangeable with the terms including “oligonucleotide”, “polynucleotide”, “DNA”, “RNA”, “gene”, “cDNA”, and “mRNA” in use.

Generally, a “template” is a polynucleotide that contains the target nucleotide sequence. In some instances, the terms “target sequence”, “template polynucleotide”, “target nucleic acid”, “target polynucleotide”, “nucleic acid template”, “template sequence”, and variations thereof, are used interchangeably. Specifically, the term “template” refers to a strand of nucleic acid on which a complementary copy is synthesized from nucleotides or nucleotide analogs through the activity of a template-dependent nucleic acid polymerase. Within a duplex, the template strand is, by convention, depicted and describe as the “bottom” strand. Similarly, the non-template strand is often depicted and described as the “top” strand. The “template” strand may also be referred to as the “sense” strand and the non-template strand as the “antisense” strand.

The term “incorporated” or “incorporation” refers to becoming a part of a nucleic acid. There is a known flexibility in the terminology regarding incorporation of nucleic acid precursors. For example, the nucleotide dGTP is a deoxyribonucleoside triphosphate. Upon incorporation into DNA, dGTP becomes dGMP, that is, a deoxyguanosine monophosphate moiety. Although DNA does not include dGTP molecules, one may say that one incorporates dGTP into DNA.

The term “initiator” refers to a mononucleoside, a mononucleotide, and oligonucleotide, a polynucleotide, or modified analogs thereof, from which a nucleic acid is to be synthesized de novo. The term “initiator” may also refer to a Xeno nucleic acids (XNA) or a peptide nucleic acid (PNA) having a 3′-hydroxyl group.

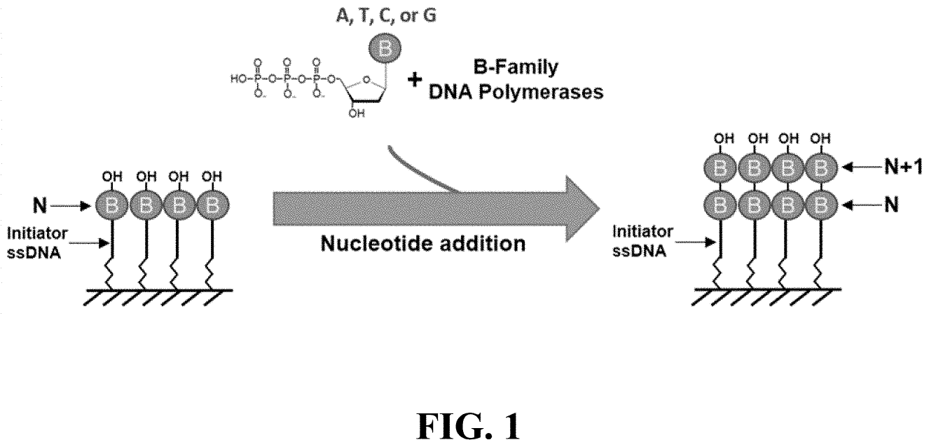

Accordingly, the present disclosure provides novel methods and kits for synthesizing nucleic acid, for example, a deoxyribonucleic acid (DNA), using newly found modified family-B DNA polymerases to solve the long-standing fundamental problems for enzymatic template-independent nucleic acid synthesis. Using the disclosed methods and kits, specific sequences of nucleotides can be synthesized de novo, base by base, in an aqueous environment, without the use of a nucleic acid template. The schematic diagram according to the invention is shown in FIG. 1. It should be noted that more nucleotide monomers (e.g. N+2, N+3, . . . N+X) can be incorporated to the initiator DNA in FIG. 1 and are omitted therein for the sake of brevity

The present disclosure provides a method for synthesizing a nucleic acid, which includes:
providing an initiator having a 3′ end having an unprotected hydroxyl group;
providing a nucleic acid polymerase having at least a conservative catalytic polymerase domain of a family-B DNA polymerase;
providing a nucleotide monomer; and
exposing the initiator to the nucleotide monomer in the presence of the nucleic acid polymerase and a metal cofactor which is a bivalent cation, and in the absence of a template, such that the nucleotide monomer is incorporated to the initiator.

The applicant surprisingly found that family-B DNA polymerases, which are well-known as template-dependent DNA polymerases, can be used to conduct template-independent nucleic acid synthesis (i.e., de novo nucleic acid synthesis). Given the unique properties of family-B DNA polymerases, such as the efficiency, thermal stability, and universality, this unexpected finding opens up a novel approach to achieve favorable outcomes of template-independent nucleic acid synthesis.

Family-B DNA polymerases (also known as type-B DNA polymerases) are replicative and repair polymerases that intrinsically have a catalytic polymerase domain and a 3′ to 5′ exonuclease domain, and can be found in bacteria, archaea, eukaryotes, and viruses. The term “catalytic polymerase domain” refers to a structural portion or region of the amino acid sequence of a protein which possesses the catalytic DNA/RNA polymerase activity of the protein, and which does not possess other catalytic activity, such as activity for editing (e.g., proofreading activity of a 3′ to 5′ exonuclease domain), activity for excision of Okazaki primers during replication, and activity for interaction with other proteins. The catalytic polymerase domains of family-B DNA polymerases have a common overall architecture, which resembles a right hand and consists of thumb, palm, and fingers subdomains. The most conserved region is the palm subdomain, which contains the catalytic site.

Although the family-B DNA polymerases and X family DNA polymerases (e.g., TdT, Pol μ, Pol λ) bind substrates in an analogous manner, their palm subdomains are not homologous to each other. As a result, they have different catalytic efficiency, replication fidelity, and biological function (Wu et al. Chem Rev. 2014 Mar. 12; 114(5): 2759-2774).

According to the disclosed invention, the family-B DNA polymerase may be sourced from bacterial family-B DNA polymerase, eukaryotic family-B DNA polymerase, archaeal family-B DNA polymerase, and viral family-B DNA polymerase.

Examples of family-B DNA polymerases include, but are not limited to, bacterial family-B DNA polymerases (e.g., Pol II), eukaryotic family-B DNA polymerases (e.g., Pol α, Pol δ, Pol ε, and Pol ζ), archaeal family-B polymerases Pol B, Pol BI, Pol BII, and Pol BIII, 9° N, Kod1, Pfu, Tgo and Vent), and viral family-B DNA polymerases (e.g., HSV-1, RB69, T4, B103, and Φ29).

In some embodiments, the family-B polymerases of archaeal family are preferably used to obtain the thermally stable and efficient enzymatic effects on nucleic synthesis.

According to the present disclosure, the initiator may be a single stranded nucleic acid, and may have a non-self complementary sequence and/or a non-self complementarity forming sequence. The term “self-complementary” means that a sequence (e.g., a nucleotide sequence or a PNA sequence) folds back on itself (i.e., a region of the sequence binds or hybridizes to another region of the sequence), creating a duplex, double strand like structure which can serve as a template for nucleic acid synthesis. Depending on how close together the complementary regions of the sequence are, the strand may form, for instance, hairpin loops, junctions, bulges or internal loops. The term “self-complementarity forming” is used to describe a sequence (e.g., a nucleotide sequence, XNA, or a PNA sequence) from which a complementary extended portion is formed when such sequence serves as a template (namely, a self-complementary sequence is formed based on such sequence serving as a template). For instance, the self-complementarity forming sequence may be “ATCC”. When the “ATCC” sequence serves as a template, an extended portion “GGAT” complementary to such sequence is formed from such sequence (i.e., a self-complementary sequence “ATCCGGAT” is formed).

In further aspects, the initiator has an unprotected nucleoside base and a 3′ hydroxyl group at the 3′ terminus. The initiator consists of a short, single strand sequence that is either a short piece of the pre-defined sequence or a universal initiator from which the pre-defined single strand product is removed. Preferably, the initiator is a DNA sequence as described above and has at least five nucleotide monomers. In an exemplary embodiment, the initiator has a DNA sequence of forty-five nucleotides.

In some embodiments, the initiator is linked, bound, immobilized or tethered to the solid support and serves as a binding site for the enzyme to catalyzes the addition of the one or more nucleotide monomer. For instance, the initiator may be directly linked to the support, or may be tethered to the support via a linker. In one embodiment, the initiator has the 5′ end linked to the solid support. Preferably, depending on user-defined synthesis of nucleic acid, the synthesized nucleic acid on the initiator is cleavable and the initiator is recyclable on the solid support.

In further embodiments, the solid support or other equivalent substrate serves as for retaining the synthesized nucleic acid during synthesis. Therefore, examples of the solid support include, but are not limited to, microarrays, beads (coated or non-coated), columns, optical fibers, wipes, nitrocellulose, nylon, glass, quartz, diazotized membranes (paper or nylon), silicones, poly formaldehyde, cellulose, cellulose acetate, paper, ceramics, metals, metalloids, semiconductive materials, magnetic particles, plastics (such as polyethylene, polypropylene, and polystyrene), gel-foaming materials (such as proteins [e.g., gelatins], lipopolysaccharides, silicates, agarose polyacrylamides, methylmethracrylate polymers), sol gels, porous polymer hydrogels, nanostructured surfaces, nanotubes (such as carbon nanotubes), and nanoparticles (such as gold nanoparticles or quantum dots), and so on.

The conservative catalytic polymerase domain of family-B DNA polymerase is an essential component for the functionality of the disclosure. The term “conservative” or “conserved” is used to describe domains containing amino acid residues that are the same among a plurality of proteins having the same structure and/or function. A region of conserved amino acid residues may be important for protein structure or function. Thus, contiguous conserved amino acid residues as identified in a three-dimensional protein may be important for protein structure or function.

For instance, Albà, Genome Biol., 2001, 2(1):reviews3002.1 to reviews3002.4 reported that family-B DNA polymerases have Regions I and II that form part of the active site of the catalytic polymerase domain, and that may respectively contain conserved amino acid residues “DT” and “SLYPS”. Region I may span amino acid residues 512 to 582, amino acid residues 513 to 582 or 583, or amino acid residues 535 to 604. Region II may span amino acid residues 375 to 441 or 442, or amino acid residues 397 to 464.

In some embodiments, the sources of the family-B DNA polymerase may include, but are not limited to, a family-B DNA polymerase of Thermococcus kodakaraensis (KOD1), a family-B DNA polymerase of Pyrococcus furiosus (Pfu), a family-B DNA polymerase of Thermococcus sp. (9° N), a family-B DNA polymerase of Thermococcus gorgonarius (Igo), and a family-B DNA polymerase of Thermococcus litoralis (Vent). KOD1, Pfu and Vent are listed as the examples of family-B DNA polymerases exhibiting thermally stable and efficient enzymatic effects on nucleic synthesis.

According to the disclosure, the nucleic acid polymerase may further have a 3′ to 5′ exonuclease domain. In some embodiments, the nucleic acid polymerase may only have the aforesaid original conservative catalytic polymerase domain without modification. In some preferred embodiments, the 3′ to 5′ exonuclease domain of the family-B DNA polymerase may be inactivated. Alternatively, the 3′ to 5′ exonuclease activity of the family-B DNA polymerase may be reduced. Still alternatively, the 3′ to 5′ exonuclease domain of the family-B DNA polymerase may remain unchanged, and specific chemical reagents or biological reagents, such as an inhibitor, may be used to inhibit the activity of the 3′ to 5′ exonuclease domain during practicing the present disclosure.

In some embodiments, the 3′ to 5′ exonuclease domain of the family-B DNA polymerase is modified in a manner comprising inactivation, attenuation, and deletion. Regarding the activity of 3′ to 5′ exonuclease, U.S. Pat. No. 8,921,043 disclosed that the archaeal DNA polymerase mutants have a double mutation in Motif 1, namely D141A and E143A, can abolish the detectible 3′ to 5′ exonuclease activity. Therefore, in the preferred embodiments, the double mutant (D141A and E143A) is introduced into the parent polymerase KOD1, Pfu, Vent to generate variants for the present invention.

According to the disclosure, the nucleotide monomer serves as the basic building block for the nucleic acid synthesis. Generally, one or more nucleotide monomers are joined to form synthetic chains of nucleotides depending on the user-defined synthesis. In some embodiments, the nucleotide monomer may be a natural nucleic acid nucleotide whose constituent elements are a sugar, a phosphate group and a nitrogen base. The sugar may be ribose in RNA or 2′-deoxyribose in DNA. Depending on whether the nucleic acid to be synthesized is DNA or RNA, the nitrogen base is selected from adenine, guanine, uracil, cytosine and thymine. Alternatively, the nucleotide monomer may be a nucleotide which is modified artificially in any of the three constituent elements. By way of example, the modification can take place at the level of the base, generating a modified product (such as inosine, methyl-5-deoxycytidine, deoxyuridine, dimethylamino-5-deoxyuridine, diamino-2,6-purine or bromo-5-deoxyuridine, and any other modified base which permits hybridization). In some embodiments, the modification occurs at the level of the sugar (for example, replacement of a deoxyribose by an analog), or at the level of the phosphate group (for example, boronate, alkylphosphate, or phosphorothioate derivatives).

According to the present disclosure, the nucleotide monomer may have a phosphate group selected from a monophosphate, a diphosphate, a triphosphate, a tetraphosphate, a pentaphosphate, and a hexaphosphate.

According to the present disclosure, the nucleotide monomer may have a removable blocking moiety. Examples of the removable blocking moiety include, but are not limited to, a 3′-O-blocking moiety, a base blocking moiety, and a combination thereof.

The nucleotide monomer having a removable blocking moiety is also referred to as a reversible terminator. Therefore, the nucleotide monomer having the 3′-O-blocking moiety is also referred to as 3′-blocked reversible terminator or a 3′-O-modified reversible terminator, and the nucleotide monomer having the base blocking moiety is also referred to as a 3′-unblocked reversible terminator or a 3′-OH unblocked reversible terminator.

As used herein, the term “reversible terminator” refers to a chemically modified nucleotide monomer. When such a reversible terminator is incorporated into a growing nucleic acid by a polymerase, it blocks the further incorporation of a nucleotide monomer by the polymerase. Such “reversible terminator” base and a nucleic acid can be deprotected by chemical or physical treatment, and following such deprotection, the nucleic acid can be further extended by a polymerase.

Examples of the 3′-O-blocking moiety include, but are not limited to, O-azidomethyl, O-amino, O-allyl, O-phenoxyacetyl, O-methoxyacetyl, O-acetyl, O-(p-toluene)sulfonate, O-phosphate, O-nitrate, O-[4-methoxy]-tetrahydrothiopyranyl, O-tetrahydrothiopyranyl, O-[5-methyl]-tetrahydrofuranyl, O-[2-methyl,4-methoxy]-tetrahydropyranyl, O-[5-methyl]-tetrahydropyranyl, and O-tetrahydrothiofuranyl, O-2-nitrobenzyl, O-methyl, and O-acyl.

Examples of the 3′-unblocked reversible terminators include, but are not limited to, 7-[(S)-1-(5-methoxy-2-nitrophenyl)-2,2-dimethyl-propyloxy]methyl-7-deaza-dATP, 5-[(S)-1-(5-methoxy-2-nitrophenyl)-2,2-dimethyl-propyloxy]methyl-dCTP, 1-(5-methoxy-2-nitropheyl)-2,2-dimethyl-propyloxy]methyl-7-deaza-dGTP, 5-[(S)-1-(5-methoxy-2-nitrophenyl)-2,2-dimethyl-propyloxyl]methyl-dUTP and 5-[(S)-1-(2-nitrophenyl)-2,2-dimethyl-propyl-oxy]methyl-dUTP.

According to the present disclosure, the base blocking moiety may be a reversible dye-terminator. Examples of the reversible dye-terminator include, but are not limited to, a reversible dye-terminator of Illulmina NovaSeq, a reversible dye-terminator of Illulmina NextSeq, a reversible dye-terminator of Illumina MiSeq, a reversible dye-terminator of Illumina HiSeq, a reversible dye-terminator of Illumina Genome Analyzer IIX, a lightning terminator of LaserGen, and a reversible dye-terminator of Helicos Biosciences Heliscope.

Since the reversible terminators are well-known to and commonly used by those skilled in the art, further details of the same are omitted herein for the sake of brevity. Nevertheless, applicable 3′-blocked reversible terminators, applicable 3′-unblocked reversible terminators, and applicable conditions for protection and deprotection (i.e., conditions for adding and eliminating the removable blocking moiety) can be found in, for example, Gardner et al. (Nucleic Acids Res. 2012 August; 40(15): 7404-7415), Litosh et al. (Nucleic Acids Res. 2011 March; 39(6): e39), and Chen et. al. (Genomics Proteomics Bioinformatics. 2013 February; 11(1): 34-40).

According to the disclosure, the initiator is exposed to the nucleotide monomer for the enzymatic reaction of synthesizing the nucleic acid. The operating temperature window of the disclosed invention is broader than currently available polymerases of template-independent nucleic acid synthesis, such as TdT (the optimum reaction/incubation temperature is 37° C. for the majority of commercially available TdT or TdT-derived enzymes). Specifically, in some embodiment, the synthesis occurs at a temperature ranging from 10° C. to 90° C.

In addition, the present disclosure provides a kit for synthesizing a nucleic acid, which includes the aforesaid initiator, the aforesaid nucleic acid polymerase, and the aforesaid nucleotide monomer. The kit is used according to the method of the present disclosure.

The present disclosure also provides another method for synthesizing a nucleic acid, which is generally similar to the aforesaid method for synthesizing a nucleic acid, except that the initiator is exposed to the at least one nucleotide monomer at a temperature ranging from 20° C. to 90° C. The temperature may range further from 30° C. to 75° C., yet further from 40° C. to 75° C., yet another further from 45° C. to 70° C., and still yet another further from 45° C. to 60° C. In exemplary embodiments, the temperature may be 30° C., 40° C., 45° C., 50° C., 60° C., 70° C. or 75° C.

The present disclosure also provides another kit for synthesizing a nucleic acid, which is generally similar to the aforesaid kit for synthesizing a nucleic acid, except that the kit is used according to the another method applied in a particular temperature range.

The another method and the another kit provided according to the present disclosure can be used for improving thermal stability and the efficiency of nucleic acid synthesis in the absence of a template.

The disclosure will be further described by way of the following examples. However, it should be understood that the following examples are solely intended for the purpose of illustration and should not be construed as limiting the disclosure in practice.

### EXAMPLES

### Example 1. Template-Independent Nucleic Acid Synthesis using Family-B DNA Polymerase of Thermococcus kodakaraensis (KOD1)

A synthesis reaction mixture was prepared using suitable amounts of the following ingredients: a single-stranded initiator that had a nucleotide sequence of SEQ ID NO: I and a 3′ end possessing an unprotected hydroxyl group and a 5′ end labeled with fluorescein amidite (FAM); deoxynucleoside triphosphates (dNTPs) serving as nucleotide monomers including dATP, dGTP, dCTP, and dTTP; a family-B DNA polymerase of Thermococcus kodakaraensis (KOD1) that has an inactivated 3′ to 5′ exonuclease domain and that is referred to as KOD1^{exo-} DNA polymerase; and a Tris-HCl buffer (pH 8.8). Specifically, the synthesis reaction mixture contained 100 nM of the initiator, 100 μM of the dNTPs, and 200 nM of KOD1^{exo-} DNA polymerase.

KOD1^{exo-} DNA polymerase was prepared as follows. A gene construct encoding a family-B DNA polymerase of KOD1 (intein-free and having a normal 3′ to 5′ exonuclease domain) was synthesized by Genomics BioSci & Tech Co. (New Taipei City, Taiwan). To obtain KOD1^{exo-} DNA polymerase, the inactivation of the conservative 3′ to 5′ exonuclease domain was achieved by changing Asp¹⁴¹ to Ala (D141A) and Glu¹⁴³ to Ala (E143A), i.e. modifying the conserved amino residues “DIE” of the conservative 3′ to 5′ exonuclease domain. Specifically, to accomplish the amino acid modification “D141A” and “E143A”, the corresponding nucleotide residues on the aforesaid gene construct were subjected to the site-directed mutagenesis using Q5 Site-directed Mutagenesis Kit (New England Biolabs, Ipswich, Mass., USA). The resulting mutagenized gene construct was expressed in BL21(DE3) cells, and the corresponding protein was purified using Akta Pure FPLC system (Cytiva Healthcare Life Sciences, Marlborough, Mass., USA) through HisTrap Q and Heparin columns sequentially. KOD1^{exo-} DNA polymerase thus obtained has an amino acid sequence of SEQ ID NO: 2.

The de novo nucleic acid synthesis reaction mixture (10 uL) was preincubated for 2 minutes at one of the following temperatures: 10° C., 20° C., 30° C., 35° C., 40° C., 45° C., 50° C., 55° C., 60° C., 70° C., 80° C., and 90° C. Subsequently, a suitable amount of Mg²⁺ serving as cofactors were added into the respective reaction mixture to initiate the template-independent nucleic acid synthesis, which was allowed to proceed for 5 minutes. The synthesis was terminated by adding 10 μL of 2× quench solution (containing 95% de-ionized formamide and 25 mM ethylenediaminetetraacetic acid (EDTA)).

The resulting synthesis reaction products were subjected to denaturation at 98° C. for 10 minutes. Subsequently, the synthesis reaction products were analyzed by 15% denaturing urea-polyacrylamide gel electrophoresis. The synthesis reaction products on the gel were visualized using Amersham Typhoon Imager (Cytiva Healthcare Life Sciences, Marlborough, Mass., USA).

### Results:

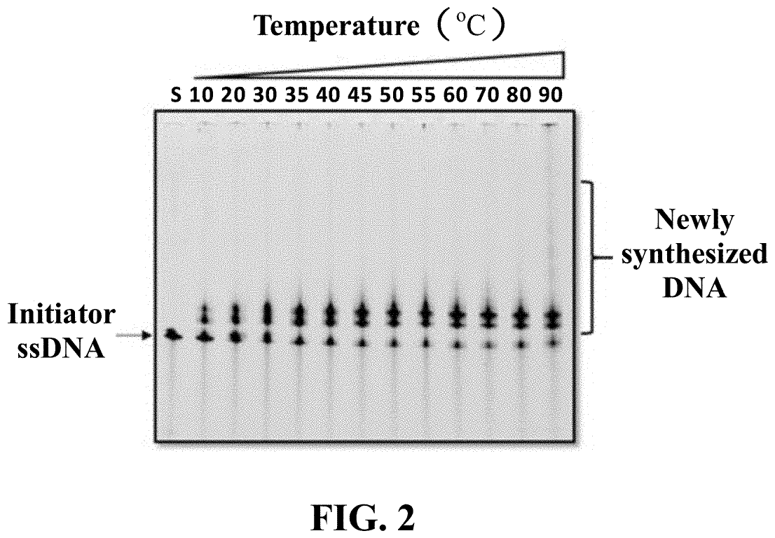

As shown in FIG. 2, KOD1^{exo-} DNA polymerase is able to perform template-independent nucleic acid synthesis at each of the temperatures tested, and thereby confirms that the family-B DNA polymerase can be used to synthesize a nucleic acid in the absence of a template de novo.

### Example 2. Template-Independent Nucleic Acid Synthesis using Family-B DNA Polymerase of Thermococcus litoralis (Vent)

Template-independent nucleic acid synthesis and its product analysis were conducted according to the procedures set forth in Example 1, except that a family-B DNA polymerase of Thermococcus litoralis (Vent) with an inactivated 3′ to 5′ exonuclease function was used. Accordingly, the 3′ to 5′ exonuclease-deficient Vent is designated as Vent^{exo-} DNA polymerase in the example. Vent^{exo-} DNA polymerase was prepared following the same procedure as that for preparing KOD1^{exo-} DNA polymerase (see Example 1), except that the intein-sequence-free gene construct encoding the family-B DNA polymerase of Thermococcus litoralis was used. The Vent^{exo-} DNA polymerase has an amino acid sequence of SEQ ID NO: 3.

### Results:

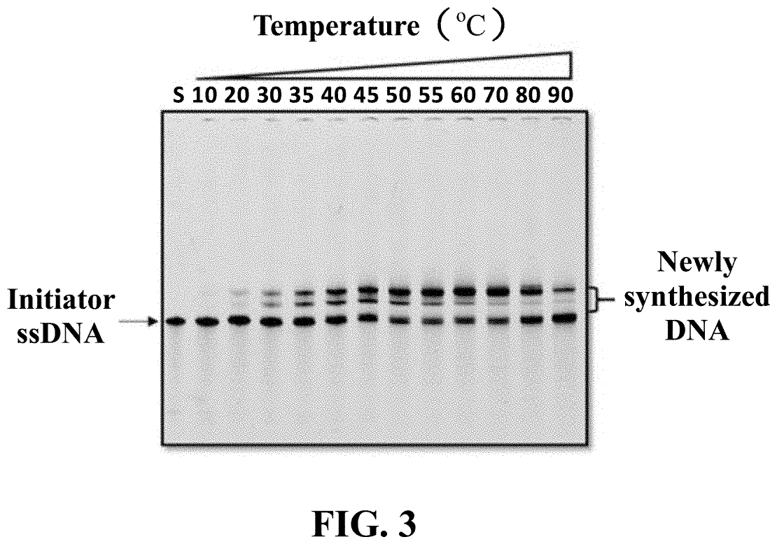

As shown in FIG. 3, Vent^{exo-} DNA polymerase is able to perform template-independent nucleic acid synthesis at each of the temperatures tested, and thereby confirms that the family-B DNA polymerase can be used to synthesize a nucleic acid in the absence of a template de novo.

### Example 3. Template-Independent Nucleic Acid Synthesis using Family-B DNA Polymerase of Pyrococcus furiosus (Pfu)

Template-independent nucleic acid synthesis and its reaction products analysis were conducted according to the procedures set forth in Example 1, except that the family-B Pfu DNA polymerase with an inactivated 3′ to 5′ exonuclease function was used. Accordingly, the 3′ to 5′ exonuclease-deficient Pfu is designated as Pfu^{exo-} DNA polymerase in the example. Pfu^{exo-} DNA polymerase was purified following the same procedure as that for preparing KOD1^{exo-} DNA polymerase (see Example 1), except that a gene construct encoding an intein-free, family-B Pfu DNA polymerase was used. The Pfu^{exo-} DNA polymerase has an amino acid sequence of SEQ ID NO: 4.

### Results:

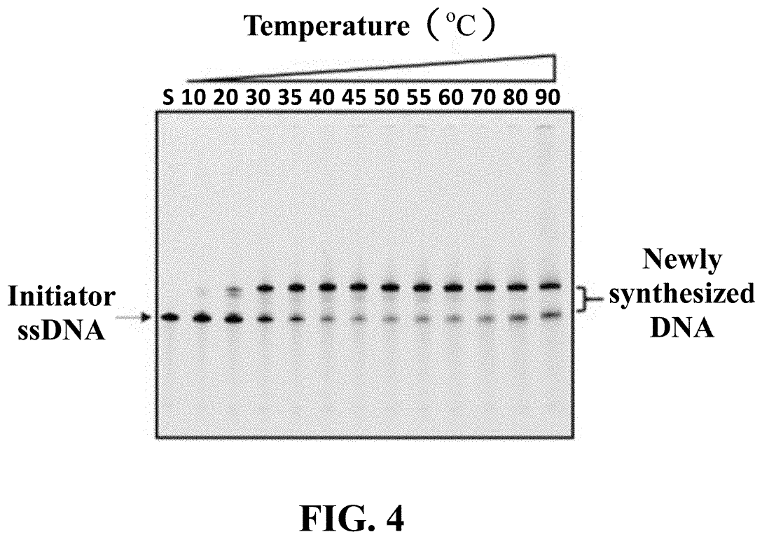

As shown in FIG. 4, Pfu^{exo-} DNA polymerase is able to perform template-independent nucleic acid synthesis at each of the temperatures tested, and thereby confirms that the family-B DNA polymerase can be used to synthesize a nucleic acid in the absence of a template de novo.

### Example 4. The Range of Reaction Temperatures for Family-B DNA Polymerases to Perform the Template-Independent Nucleic Acid Synthesis

As demonstrated in the above examples, the template-independent nucleic acid synthesis by family-B DNA polymerases can be performed at a broad range of reaction temperatures ranging from 10° C. to 90° C.

A. Determination of a more Favorable Temperature Range for Template-Independent Nucleic Acid Synthesis by Family B DNA Polymerases

To narrow down a more favorable temperature range for family-B KOD1^{exo-} DNA polymerase to efficiently synthesize nucleic acid de novo, the experiment was conducted as in example 1, except that the temperatures were set at 20° C., 30° C., 40° C., 45° C., 50° C., 60° C., 70° C., 75° C., 80° C., and 90° C., respectively. Additionally, the synthesis reaction was allowed to proceed for 30 minutes to enhance the differences of each reaction at different temperature.

The resulting synthesis reaction products were subjected to denaturation at 95° C. for 10 minutes. The reaction products were analyzed by 20% polyacrylamide gel electrophoresis (PAGE) containing 8 M urea. The image of the gel was visualized by Amersham™ Typhoon laser scanner (Cytiva Lifesciences, Marlborough, Mass., USA).

### Results:

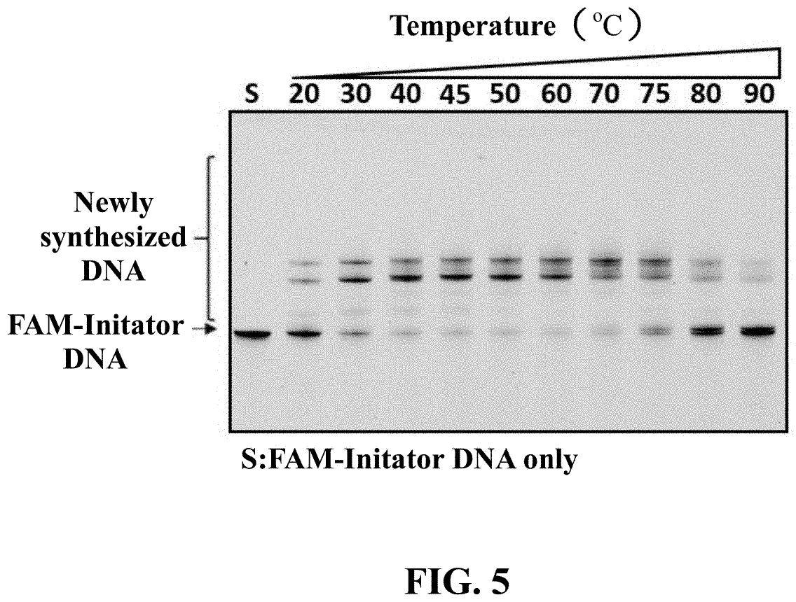

Referring to FIG. 5, KOD1^{exo-} DNA polymerase is able to perform template-independent nucleic acid synthesis in a longer reaction period at the temperature between 20° C. and 90° C. Furthermore, more synthesis reaction products were produced by KOD1^{exo-} DNA polymerase at between 30° C. and 75° C.

### B. Determination of Relative Activity for Template-Independent Nucleic Acid Synthesis by Family B DNA Polymerases at Different Reaction Temperatures

The relative nucleic acid synthesis activities of KOD1^{exo-} DNA polymerase at different reaction temperatures ranging from 20° C. to 90° C. were measured by quantifying the intensity of each fragments using ImageJ software (NIH, Bethesda, USA) and calculating their relative ratios to the starting initiator DNA band on the urea-PAGE gel. Specifically, the relative nucleic acid synthesis activities of family B DNA polymerases at different reaction temperatures were determined by comparing the image intensity of the remaining initiator DNA band in each reaction on the gel to the non-reactive or starting initiator DNA band, as exemplified in Section A.

### Results:

The relative nucleic acid synthesis activities of KOD1^{exo-} DNA polymerase at different reaction temperatures are listed in Table 1 below.

TABLE 1

| Temperature | Relative |
|---|---|
| (° C.) | activity (%) |
| 20 | 33.8 |
| 30 | 67.1 |
| 40 | 84.2 |
| 45 | 88.9 |
| 50 | 91.1 |
| 60 | 91.6 |
| 70 | 89.2 |
| 75 | 74.4 |
| 80 | 28.5 |
| 90 | 6.2 |

As shown in Table 1, the template-independent nucleic acid synthesis of KOD1^{exo-} DNA polymerase can be performed at a temperature between 20° C. and 80° C., and more favorable at a temperature between 30° C. and 75° C. Furthermore, the synthesis activity of KOD1^{exo-} DNA polymerase is more efficient at a temperature between 40° C. and 75° C., and even more robust at a temperature between 45° C. and 70° C.

C. Evaluation of Template-Independent Nucleic Acid Synthesis Activity of Terminal Deoxynucleotidyl Transferase (TdT) at Favorable Reaction Temperatures of family-B KOD1^{exo-} DNA Polymerase

The template-independent nucleic acid synthesis activity of conventional Tdt was determined as in example 1, except that the reaction temperatures were set at 45° C., 50° C., 60° C., 70° C., 75° C., 80° C., and 90° C., respectively, and the Tdt buffer was used. Both TdT and its reaction buffer were obtained from New England Biolabs (ipswitch, Mass., USA).

### Results:

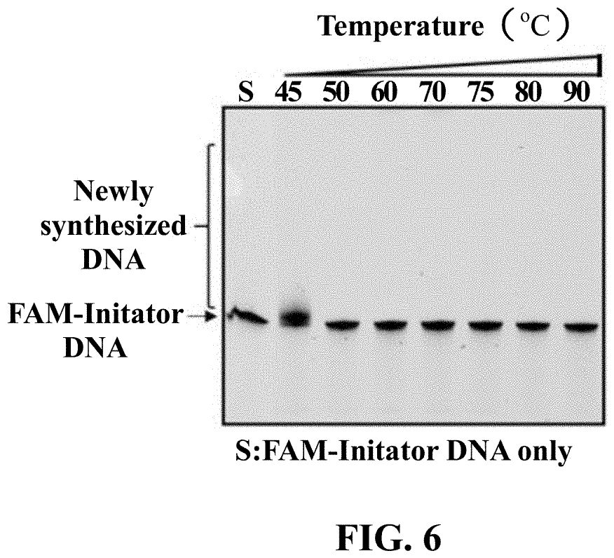

Referring to FIG. 6, the template-independent nucleic acid synthesis activity of TdT is significantly reduced at 45° C. and completely diminished at the temperature equal to or higher than 50° C. On the contrary, as shown in FIG. 5, KOD1^{exo-} DNA polymerase is able to efficiently synthesize nucleic acid at the temperature equal to or higher than 45° C., manifesting that KOD1^{exo-} DNA polymerase is thermally stable and more effective in template-independent nucleic acid synthesis than the conventional terminal deoxynucleotidyl transferase (Tdt) at higher reaction temperatures (i.e. 45° C. to 90° C.).

### D. Determination of a Favorable Reaction Temperature Range for Other Family-B DNA Polymerases

As shown in FIG. 5 and Table 1, the favorable reaction temperatures for template-independent nucleic acid synthesis activity of KOD1^{exo-} DNA polymerase are between 30° C. and 75° C. Furthermore, the nucleic acid synthesis activity of KOD1^{exo-} DNA polymerase is more efficient at a temperature between 40° C. and 75° C., and even more robust at a temperature between 45° C. and 70° C. To confirm whether the same reaction temperature ranges can also be applied to other family-B DNA polymerases, the Vent^{exo-} and Pfu^{exo-} DNA polymerase respectively mentioned in Examples 2 and 3 were tested according to the procedure described in section A of this example. The template-independent nucleic acid synthesis reaction for Vent^{exo-} or Pfu^{exo-} DNA polymerase was performed at 30° C., 40° C., 45° C., 50° C., 60° C., 70° C., 75° C., 80° C., and 90° C., respectively.

### Results:

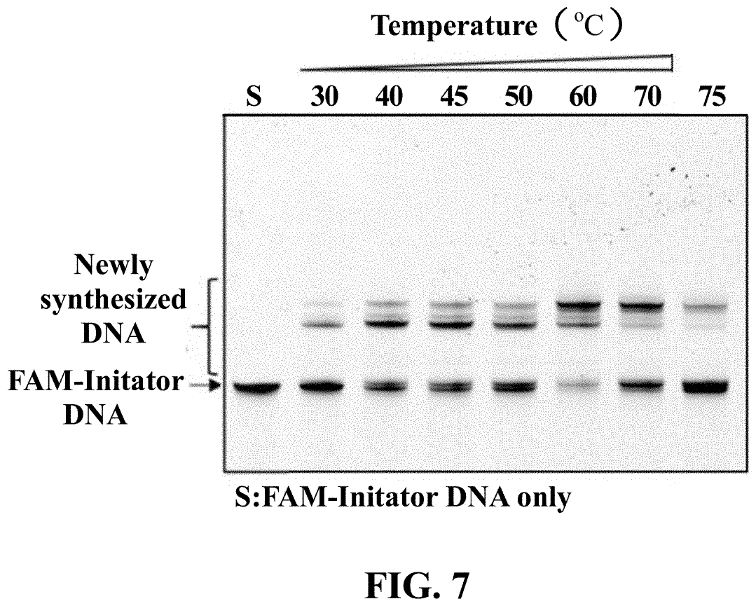

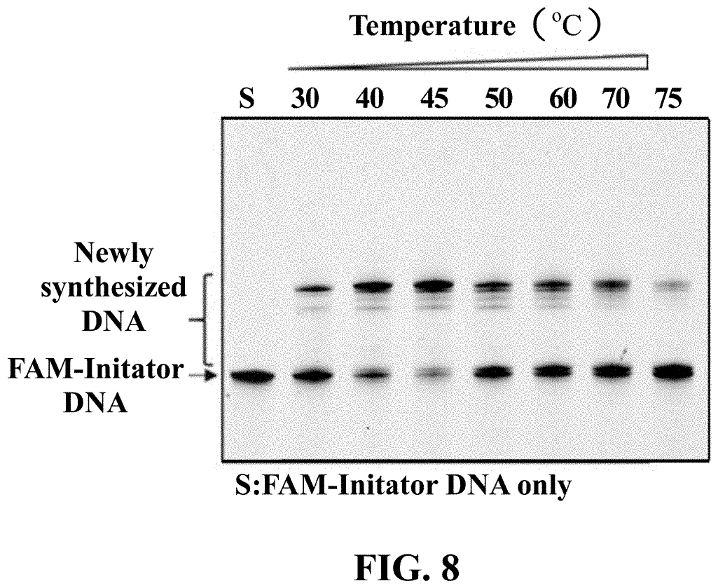

Referring to FIG. 7, Vent^{exo-} DNA polymerase is confirmed to be able to perform template-independent nucleic acid synthesis at the temperature range between 30° C. and 75° C., and effectively perform template-independent nucleic acid synthesis at the temperature range between 40° C. and 75° C. and at the even more favorable temperature range between 45° C. to 70° C. Likewise, referring to FIG. 8, Pfu^{exo-} DNA polymerase is confirmed to be able to perform template-independent nucleic acid synthesis at the temperature range between 30° C. and 75° C., and effectively perform template-independent nucleic acid synthesis at the temperature range between 40° C. and 75° C. and the even more favorable temperature range between 45° C. to 70° C.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for synthesizing a nucleic acid, comprising:
providing an initiator having an unprotected nucleoside base and a 3′ hydroxyl group at a 3′ terminus;
providing a nucleic acid polymerase having at least one conservative catalytic polymerase domain of a family-B DNA polymerase;
providing at least one nucleotide monomer; and
exposing the initiator to the at least one nucleotide monomer in the presence of the nucleic acid polymerase, at a temperature ranging from 20° C. to 90°, and in the absence of a template, such that the at least one nucleotide monomer is incorporated to the initiator to form the nucleic acid.

2. The method of claim 1, wherein the temperature ranges from 30° C. to 75° C.

3. The method of claim wherein the temperature ranges from 40° C. to 75° C.

4. The method of claim 1, wherein the temperature ranges from 45° C. to 70° C.

5. The method of claim wherein the temperature ranges from 45° C. to 60° C.

6. The method of claim 1, wherein the initiator is a single stranded nucleic acid.

7. The method of claim 1, wherein the initiator is linked to a solid support and has a 5′ end linked to the solid support.

8. The method of claim 7, wherein the solid support is selected from the group consisting of a microarray, a bead, a column, an optical fiber, a wipe, nitrocellulose, nylon, glass, quartz, a diazotized membrane, a silicone, polyformaldehyde, celluloses, cellulose acetate, paper, a ceramic, a metal, a metalloid, a semiconductor material, a magnetic particle, a plastic, a gel-forming material, a gel, a nanostructure surface, a nanotube, a nanoparticle, and a combination thereof.

9. The method of claim 1, wherein the family-B DNA polymerase is selected from the group consisting of family-B DNA polymerase of Thermococcus kodakaraensis (KOD1), family-B DNA polymerase of Pyrococcus furiosus (Pfu), family-B DNA polymerase of Thermococcus sp. (9° N), family-B DNA polymerase of Thermococcus gorgonarius (Tgo) , and family-B DNA polymerase of Thermococcus litoralis (Vent).

10. The method of claim 1, wherein the nucleic acid polymerase further has a 3′ to 5′ exonuclease domain.

11. The method of claim 10, wherein the 3′ to 5′ exonuclease domain of the family-B DNA polymerase is modified in a manner comprising inactivation, attenuation, and deletion.

12. The method of claim 11, wherein the 3′ to 5′ exonuclease domain of the family-B DNA polymerase has at least one mutation selected from the group consisting of D141A, E143A, and a combination thereof.

13. The method of claim 1, wherein the nucleotide monomer has a phosphate group comprising a monophosphate, a diphosphate, a triphosphate, a tetraphosphate, a pentaphosphate, a hexaphophate, and a combination thereof.

14. The method of claim 1, wherein the nucleotide monomer has a removable blocking moiety selected from the group consisting of a 3′-O-blocking moiety, a base blocking moiety, and a combination thereof.

15. A kit for synthesizing a nucleic acid, comprising:
an initiator having an unprotected nucleoside base and a 3′ hydroxyl group at a 3′ terminus;
a nucleic acid polymerase having at least one conservative catalytic polymerase domain of a family-B DNA polymerase; and
at least one nucleotide monomer;
wherein the kit is used through exposing the initiator to the at least one nucleotide monomer in the presence of the nucleic acid polymerase, at a temperature ranging from 20° C. to 90°, and in the absence of a template, such that the at least one nucleotide monomer is incorporated to the initiator to form the nucleic acid.

16. The kit of claim 15, wherein the initiator is a single stranded nucleic acid.

17. The kit of claim 15, wherein the family-B polymerase is selected from the group consisting of family-B DNA polymerase of Thermococcus kodakaraensis (KOD1), a family-B DNA polymerase of Pyrococcus furiosus (Pfu), family-B DNA polymerase of Thermococcus sp. (9° N), family-B DNA polymerase of Thermococcus gorgonarius (Tgo), and family-B DNA polymerase of Thermococcus litoralis (Vent).

18. The kit of claim 15, wherein the nucleic acid polymerase further has a 3′ to 5′ exonuclease domain.

19. The kit of claim 17, wherein the 3′ to 5′ exonuclease domain of the family-B DNA polymerase is modified in a manner comprising inactivation, attenuation, and deletion.

20. The kit of claim 18, wherein the 3′ to 5′ exonuclease domain of the family-B DNA polymerase has at least one mutation selected from the group consisting of D141A, E143A, and a combination thereof.

21. The kit of claim 15, wherein the nucleotide monomer has a removable blocking moiety selected from the group consisting of a 3′-O-blocking moiety, a base blocking moiety, a base blocking moiety, and a combination thereof.


SEQUENCE LISTING (4 sequences)

SEQ ID NO: 1 - DNA, 45 bp, Artificial Sequence
  Initiator for template-independent nucleic acid synthesis
ctcggcctggcacaggtccgttcagtgctgcggcgaccaccgagg

SEQ ID NO: 2 - protein, 774 aa, Artificial Sequence
  KOD1(exo-) DNA polymerase
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 3 - protein, 774 aa, Artificial Sequence
  Vent(exo-) DNA polymerase
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 4 - protein, 775 aa, Artificial Sequence
  Pfu(exo-) DNA polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS